(19) United States
(12) Patent Application Publication
(10) Pub. No.: US 2011/0123545 A1
(43) Pub. Date: May 26, 2011

(54) COMBINATION OF VEGFR2 AND IGF1R INHIBITORS FOR THE TREATMENT OF PROLIFERATIVE DISEASES

(75) Inventors: H. Nicholas Marsh, Charlestown, MA (US); Irvith M. Carvajal, Somerville, MA (US); Marco M. Gottardis, Princeton, NJ (US); Joan M. Carboni, Yardley, PA (US); Bryan C. Barnhart, Philadelphia, PA (US); Ricardo M. Attar, Lawrenceville, NJ (US)

(73) Assignee: Bristol-Myers Squibb Company

(21) Appl. No.: 12/953,541

(22) Filed: Nov. 24, 2010

Related U.S. Application Data
(60) Provisional application No. 61/264,115, filed on Nov. 24, 2009.

(51) Int. Cl. A61K 39/395; A61P 35/00

(52) U.S. Cl. 424/158.1

(57) ABSTRACT

Methods for increasing endothelial cell apoptosis under hypoxic conditions in a patient undergoing a treatment that comprises an IGF1R inhibitor, the method comprising administering to the patient a VEGFR2 inhibitor

This application claims the benefit of U.S. Provisional Application No. 61/264,115 filed Nov. 24, 2009, whose contents are hereby incorporated by reference.

### FIELD OF THE INVENTION

This invention relates to the fields of oncology and improved therapy regimens.

### BACKGROUND OF THE INVENTION

Inhibition of VEGFR2 and/or IGF1R is attractive for the treatment of proliferative diseases, including cancer.

WO2005/56764 discloses molecules binding to VEGFR2. WO2008/066752 discloses molecules binding to IGF1R. Moreover, WO2008/97497 discloses molecules that affect the VEGF pathway blockade, WO2009/025806 discloses molecules for treatment of metastatic tumors, and WO2009/073115 discloses combinations of a VEGFR2 inhibitor and an mTOR inhibitor.

WO2009/142773 hereby incorporated by reference discloses (i) monospecific molecules to VEGFR2 or IGF1R and (ii) bispecific molecules to VEGR2 and IGF1R. This application discloses bispecific molecules that were designed to bind and inactivate VEGFR2 and IGF1R, providing a unique opportunity to inhibit these key growth factor receptors with a single agent. These bispecific molecules bind VEGFR2 and IGF1R with high affinity and specificity, are able to inhibit receptor activation both in vitro and in vivo, are effective in vivo in elevating IGF1R and VEGFR2 biomarkers, and are active in IGF and VEGF-dependent mouse tumor models.

There remains a need for methods of treating proliferative diseases (e.g., cancer), as well as methods for identifying molecules that are useful for the treatment of proliferative diseases (e.g., cancer).

### SUMMARY OF THE INVENTION

The present invention provides data that inhibiting the IGF1R axis alone can cause growth inhibition and increases in apoptosis in endothelial cells under hypoxic conditions, and that inhibiting both IGF1R and VEGFR2 has a greater impact on endothelial cells (including increasing endothelial cell apoptosis under hypoxic conditions) than inhibiting VEGFR2 or IGF1R alone. It is also shown that inhibiting VEGFR2 and IGF1R together can affect endothelial cell morphology as measured by vascular corrosion casting.

In one aspect, the present invention comprises a method for increasing endothelial cell apoptosis under hypoxic conditions in a patient undergoing a treatment that comprises an IGF1R inhibitor, said method comprising administering to said patient a VEGFR2 inhibitor.

The IGF1R inhibitor and VEGFR2 inhibitor can be co-administered to said patient. In one aspect, the IGF1R inhibitor and VEGFR2 inhibitor are not derived from 10Fn3.

In another aspect, the present invention comprises a method for identifying a molecule for the treatment of a proliferative disease comprising the steps of: (i) identifying an IGF1R inhibitor; (ii) identifying a VEGFR2 inhibitor; and (iii) combining the properties of said first agent and send second agent in a molecule that is effective for the treatment of said proliferative disease.

### DETAILED DESCRIPTION OF THE INVENTION

The present invention provides methods of treating proliferative diseases (e.g., cancer), as well as methods for identifying molecules that are useful for the treatment of proliferative diseases (e.g., cancer).

IGF1R inhibitors useful for practicing the present invention are well known in the art. In one aspect, the IGF1R inhibitor is a small molecule compound (e.g., BMS-754807). In another aspect, the IGF1R inhibitor is a biologic (e.g., an antibody, or an Adnectin molecule derived from 10Fn3).

VEGFR2 inhibitors useful for practicing the present invention are well known in the art. In one aspect, the VEGFR2 inhibitor is a small molecule compound. In another aspect, the VEGFR2 inhibitor is a biologic (e.g., an antibody, or an Adnectin molecule derived from 10Fn3).

### EXAMPLES

The 10Fn3-based binders used in Examples 1-5 were as follows: “VEGFR2 binder” or “V binder” is Peg-V2BShort as disclosed in WO2009/142773; “IGF1R binder” or “I binder” is 385A08 with Cys tail as disclosed in WO2009/142773; and “bispecific VEGFR2/IGF1R binder” or “V/I binder” is 385A08-Fn-V2B-Cys pegylated without His tag (Examples 1, 3, and 4) or with His tag (Examples 2 and 5) as disclosed in WO2009/142773.

### Example 1

### Activation and Signaling Activity under Hypoxia in Tumor Cell-Based Assays

Target effects of VEGFR2, IGF1R, and VEGFR2/IGF1R binders were evaluated in Rh41 Rhabdomyosarcoma cells in hypoxic (0.5% Oxygen (O₂)) conditions or normoxic conditions by immunoblotting. Cells were seeded at 1×106 cells in 6-well plates in RPMI with 10% FCS and incubated overnight at 37° C. in hypoxia (with 5% carbon dioxide and 94.5% nitrogen in an InVivo2 workstation (Ruskinn Inc., Cincinnati, Ohio)) or normoxia. The next day, cells were serum-starved in RPMI media with 0.3% bovine serum albumin for 6 hours (hypoxia-equilibrated media was used in hypoxic conditions) and cells were either maintained in ambient O₂ or incubated at 0.5% O₂. Indicated 10Fn3-based binders were added at 20 nM final concentration for two hours. IGF-1 was then added as indicated for the final 10 minutes of culture at 50 ng/mL. Cells were then lysed in TTG (1% Triton X-100, 5% Glycerol, 150 mM NaCl, 20 mM Tris-HC1, pH7.6, 1 mM EDTA with protease inhibitor tablets (Roche, Indianapolis, Ind.) and Phosphatase inhibitor cocktail (Sigma-Aldrich Corp., St. Louis, Mo.)) and total protein was quantified with the BCA protein assay (Pierce, Waltham, Mass.). Levels of total IGF1R and the phosphorylation state of the IGF1R, phosphorylated AKT (Serine 473), phosphorylation state of p70S6K, HIF1α, and cleaved caspase-3, were detected by SDS-PAGE analysis of 30 micrograms of total protein followed by transfer of proteins to nitrocellulose and immunoblotting with specific antibodies. Blots were probed with GAPDH to demonstrate equal loading of each sample.

The binders demonstrated a decrease in phosphorylation state of IGF1R. Hypoxic incubation elicited an increase in HIF1α and a reduction in phosphorylated p70S6K which was further reduced by incubation with IGF1R binder. Treatment with these binders in hypoxia caused a greater production of cleaved caspase-3 than in normoxia which was further enhanced by IGF1R binder (V/I binder inhibited pAKT better under hypoxia than normoxia; there was more apoptosis under hypoxia than normoxia by V/I binder and I binder).

### Example 2

### Activation and Signaling Activity under Hypoxia in Cell-Based Assays with Endothelial Cells

Target effects of various VEGFR2, IGF1R, or VEGFR2/IGF1R binders were evaluated in human microvascular endothelial cells (HMVEC) in hypoxic (0.5% O₂) or normoxic conditions by immunoblotting. Cells were seeded at 1×106 cells in 6 cm plates and incubated overnight in EBM2 with EGM-2 MV SingleQuots growth factor cocktail (Lonza, Basel, Switzerland) media at 37° C. in hypoxia (0.5% O₂ in an InVivo2 workstation (Ruskinn Inc., Cincinnati, Ohio) with 5% Carbon dioxide and 94.5% Nitrogen, or normoxia with 5% CO₂) or normoxia. The next day, treatments were initiated, and cells were either maintained in ambient O₂ or incubated in hypoxia under serum-starved conditions with EBM2 with 0.3% bovine serum albumin in both cases (hypoxia-equilibrated medium was used for hypoxic experiments). Indicated 10Fn3-based binders or anti-IGF1R antibody were added at 20 nM final concentration for two hours. IGF-1 and VEGF were then added as indicated for the final 10 minutes of culture at 50 ng/mL each. Cells were then lysed in TTG (1% Triton X-100, 5% Glycerol, 150 mM NaCl, 20 mM Tris-HC1, pH7.6, 1 mM EDTA with protease inhibitor tablets (Roche, Indianapolis, Ind.) and Phosphatase inhibitor cocktail (Sigma-Aldrich Corp., St. Louis, Mo.)) and total protein was quantified with the BCA protein assay (Pierce, Waltham, Mass.). Levels of total IGF1R and VEGFR2 and the phosphorylation state of the each receptor, phosphorylated AKT (Serine 473), phosphorylation state of p70S6K, HIF1α, and cleaved caspase-3, were detected by SDS-PAGE analysis of 30 micrograms of total protein followed by transfer of proteins to nitrocellulose and immunoblotting with specific antibodies. Blots were probed with GAPDH to demonstrate equal loading of each sample.

The IGF1R binder demonstrated a decrease in phosphorylation state of IGF1R, and the VEGFR2 binder demonstrated a decrease in phosphorylated VEGFR2. Hypoxic incubation elicited an increase in HIF1α which was reduced by 48 hours in hypoxia. Phosphorylated p70S6K was reduced in hypoxia and further reduced by incubation with IGF1R and VEGFR2 binders. Phosphorylated AKT was reduced by treatment with IGF1R binder and was more reduced following this treatment in hypoxia. Treatment with IGF1R and VEGFR2 binders induced caspase-3 processing (indicative of apoptosis) which was enhanced in hypoxia, suggesting that these pathways are protective in cells exposed to hypoxic conditions. (I binder inhibited pIGF1R better under hypoxia than normoxia; more apoptosis under hypoxia than normoxia by V/I binder and I binder)

### Example 3

### Apoptosis induction in HMVEC

Effect of hypoxia and the various VEGFR2, IGF1R, or VEGFR2/IGF1R binders on induction of apoptosis were evaluated in HMVEC endothelial cells with or without incubation in hypoxic conditions (0.5% Oxygen (O₂)) by Annexin V and propidium iodide assay. Cells were seeded at 1×105 cells per well in 6-well plates in EBM2 with EGM-2 MV SingleQuots growth factor cocktail (Lonza, Basel, Switzerland) media and incubated 37° C. in 5% CO₂ overnight to adhere. Cells were treated with indicated reagents and incubated for 48 hours in hypoxic (3% O₂) conditions in an InVivo2 workstation (Ruskinn Inc., Cincinnati, Ohio) with 5% carbon dioxide and 94.5% nitrogen, or under normoxic conditions. Treatment with VEGFR2, IGF1R or VEGFR2/IGF1R binders at 1 micromolar final concentration was as indicated. Cells were then gently harvested with 0.05% Trypsin (Gibco/Invitrogen, Camarillo, Calif.), washed twice, and evaluated with the ApoTarget kit (Invitrogen, Camarillo, Calif.). Apoptosis was evaluated by assessment of Annexin V positivity on a FACSCalibur flow cytometer using FL-1 channel for Annexin V-FITC and FL-2 channel for propidium iodide without gating on cell populations. Percentage of apoptosis was determined using FloJo software (Tree Star, Inc., Ashland, Oreg.) and was based on total Annexin V positive cells. Apoptosis over control was assessed by subtracting apoptosis in normoxic samples from hypoxic samples.

Incubation of HMVEC in 3% O₂ for 48 hours induced 15-20% specific apoptosis. Treatment with VEGFR2 binder induced a small increase in apoptosis. IGF1R binder induced a similar increase in apoptosis in hypoxic conditions over background, indicating that IGF1R blockade results in reduced viability in endothelial cells exposed to hypoxia. Treatment with VEGFR2/IGF1R binder induced ˜17% specific apoptosis over control in hypoxic conditions. These data suggest that both IGF1R and VEGFR2 pathways in HMVEC cells act as pro-survival pathways under hypoxic conditions.

### Example 4

### Activation and Signaling Activity under Hypoxia in Cell-Based Assays with Endothelial Cells (Small Molecule IGF-1R Inhibitor Combinations)

Target effects of the various VEGFR2, IGF1R or VEGFR2/IGF1R binders, IGF-1R inhibitor BMS-754807, and antagonistic anti-IGF-1R antibody MAB309 were evaluated in human microvascular endothelial cells (HMVEC) in hypoxic (0.5% O₂) or normoxic conditions by immunoblotting. Cells were seeded at 1×106 cells in 6 cm plates and incubated overnight in EBM2 with EGM-2 MV SingleQuots growth factor cocktail (Lonza, Basel, Switzerland) media at 37° C. in hypoxia (0.5% O₂ in an InVivo2 workstation (Ruskinn Inc., Cincinnati, Ohio) with 5% Carbon dioxide and 94.5% Nitrogen, or normoxia with 5% CO₂) or normoxia. The next day, treatments were initiated, and cells were either maintained in ambient O₂ or incubated in hypoxia under serum-starved conditions with EBM2 with 0.3% bovine serum albumin in both cases (hypoxia-equilibrated medium was used for hypoxic experiments). Indicated 10Fn3-based binders or anti-IGF1R antibody were added at 20 nM final concentration for two hours. IGF-1 and VEGF were then added as indicated for the final 10 minutes of culture at 50 ng/mL each. Cells were then lysed in TTG (1% Triton X-100, 5% Glycerol, 150 mM NaCl, 20 mM Tris-HCl, pH7.6, 1 mM EDTA with protease inhibitor tablets (Roche, Indianapolis, Ind.) and Phosphatase inhibitor cocktail (Sigma-Aldrich Corp., St. Louis, Mo.)) and total protein was quantified with the BCA protein assay (Pierce, Waltham, Mass.). Levels of total IGF1R and VEGFR2 and the phosphorylation state of the each receptor, phosphorylated AKT (Serine 473), phosphorylation state of p70S6K, and HIF1α were detected by SDS-PAGE analysis of 30 micrograms of total protein followed by transfer of proteins to nitrocellulose and immunoblotting with specific antibodies. Blots were probed with GAPDH to demonstrate equal loading of each sample.

The IGF1R binder demonstrated a decrease in phosphorylation state of IGF1R, and the VEGFR2 binder demonstrated a decrease in phosphorylated VEGF-R2. Hypoxic incubation elicited an increase in HIF1α which was reduced by 48 hours in hypoxia. Phosphorylated p70S6K was reduced in hypoxia and further reduced by incubation with IGF1R and VEGFR2 binders. Phosphorylated AKT was reduced by treatment with IGF1R binder and was more reduced following this treatment in hypoxia. Treatment with IGF1R and VEGFR2 binders resulted in a decrease of phosphorylated p70S6K, AKT and phosphorylated VEGFR2 and IGF1R. Co-treatment with VEGFR2 binder and BMS-754807 resulted in a decrease in phosphorylated p70S6K, AKT and phosphorylated VEGFR2 and IGF1R. Hypoxia enhanced the inhibition of AKT and p70S6K.

### Example 5

### Evaluation and Quantitation of Early Vascular Effects in an A673 Ewing Sarcoma Xenograft Model

The primary objective of this study was to evaluate the vascular effects of a VEGFR2/IGF1R binder, IGF1R binder, and VEGFR2 binder in comparison to untreated tumors in a Ewing sarcoma xenograft model. Given the reported low sensitivity of this Ewing sarcoma model to bevacizumab (anti-VEGF antibody), a group of bevacizumab-treated tumors was included to determine whether changes on vascular morphology can be observed under suboptimal conditions of tumor growth inhibition.

Experimental Design:

75 Female NCR-nude A673-tumor bearing mice were assigned into five groups, 15 mice per group when their tumors reached a tumor volume average of 300 mm3 by caliper measurement:

Group 1=Vehicle

Group 2=VEGFR2 binder (80 mg/kg three times a week)

Group 3=IGF1R binder (80 mg/kg three times a week)

Group 4=VEGFR2 binder+IGF1R binder (80 mg/kg each)

Group 5=VEGFR2/IGF1R binder (160 mg/kg three times a week)

Tumors were collected at the end of 14 days of treatment for tumors for vascular corrosion casting and at the end of 7 days of treatment for light microscopy analysis. Treatments were administered intraperitoneally. The doses selected for the individual 10Fn3 binders were stoichiometrically equivalent to that of the tandem 10Fn3 binder dose. Per group, 10 mice were collected for generation of tumor casts, and 5 mice were processed for tumor light microscopy analysis. An additional group of five A673-tumor bearing mice were selected for bevacizumab treatment (10 mg/kg BW), with larger tumors (tumor volume average of 800 mm3), were only used for corrosion casting comparisons. No bevacizumab-treated tumors were evaluated for light microscopy. The purpose of this group was to address the question of whether vascular changes can be observed in the absence of tumor growth inhibition effects with an anti-VEGF blocker (bevacizumab).

Vascular Corrosion Casting and Light Microscopy:

For vascular corrosion casting, euthanasized mice were flushed with bodywarm saline, fixed with glutaraldehyde and injected with PU4ii resin mixed with hardener (40% solvent) and blue dye obtained from VasQTec (Zurich, Switzerland). After overnight curing of the resin the whole mice were individually bagged and frozen at −18° C. Tumor cast processing was done according to published standard protocols. The tumors that did not undergo microvascular corrosion casting were dissected out, and cut into 2 sections. One half was fixed with formaldehyde for paraffin embedding. The remaining tissue was snap frozen for immunohistochemistry.

Morphometric analysis: The tumor vascular architecture, volume, and capillary density were evaluated by means of complementary 2D and 3D morphologic and morphometric analyses. 3D microvascular corrosion casting provides quantitative data on tumor microvascular architecture and pattern formation.

2D Evaluation of Microvessel Density:

Conventional histology was used for histopathology and calculation of the amount of necroses. Anti-CD31 stains were used for 2D assessment of vascular surface areas. Microvessel densities were determined in frozen sections after anti-CD 31 staining. From each tumor section, two to five micrographs of representative areas were chosen and evaluated with a Weibel grid. All micrographs were taken with an objective magnification of ×5 (Zeiss Axiophot) and displayed to full TFT-screen size. The final magnification covered an area of 3.28025 mm2 A Weibel square grid was superimposed on the micrographs, consisting of boxes measuring 0.445×0.415 cm. The whole grid consisted of 52.2×34.3 boxes, in total 1790 boxes. The size of an individual grid box covered thus 1832 μm2 (40×45.8 μm). All cases, in which a CD31 positive cell or a vessel lumen coincided with the upper left corner of an individual box, were counted as positive events. The total amount of positive crossings divided by the total area of the evaluated grid (=all boxes covering tumor tissue) gives the percentual vascular surface area.

Three positive events are seen in a 10×10 grid (=100 boxes). Only the highlighted dots cross the upper left corner of a square and are counted as positive results; the other dots do not cross the upper left corner of a grid box. In the first set of evaluation, all tumor areas were counted. Only blanks were excluded. In a second step, all necroses as assessed by interactive morphometry were additionally excluded in order to determine the percentual vessel density in the viable tissue only.

Cast Vascular Volume: After maceration of the injected tumors, all casts were weighed in order to calculate the functional vascular volume.

Microvascular architecture: qualitative aspects: The evaluable casts were macerated, rinsed, freeze dried, cut and mounted on specimen holders, sputtered with gold in argon atmosphere and looked on with a Philips ESEM scanning electron microscope. Stereopairs with a tilt angle of 6° were gathered using a eucentric specimen holder. The stereopairs were used for morphometry of parameters defining the architecture of the microvascular unit.

Microvascular architecture: quantitative data: From each tumor series of stereopairs with tilt angles of 6° were made for quantitative analyses. The stereo pairs were color coded and reconstructed as anaglyphic images. With the known tilt angle, calculations of individual points marked interactively in both images of each stereopair were carried out using macros defined for the Kontron KS 300 software. For definition of the dimensions of the microvascular unit, the intervascular distances as well as the interbranching distances (=vessel segment lengths) were calculated. In addition, the vessel diameters were assessed. A total of approximately 30.000 measurements were carried out. Due to the high numbers of individual measurements graphically minor differences are highly significant.

Results:

(I) Tumor Weights from Histology-Cohort Mice

Tumors from histology-cohort mice did not show significant differences in size or weight despite the clear tendency towards lower values in all 10Fn3 treatment groups. Given the short term treatment duration for this cohort, these results are not surprising.

(II) Tumor Weights from Vascular Corrosion Casting-Cohort Mice

Significant differences in tumor weight were seen between vehicle and VEGFR2/IGF1R binder, VEGFR2 binder, and the combo groups. The longer duration of treatments for this cohort may account for the observed differences, but in addition, these groups have in common the inhibition of VEGFR2 by VEGFR2 binder, which more importantly relates to tumor growth inhibition effects in this model. Given that the IGF1R binder did not show significant differences vs. vehicle, it is possible that under this length of treatment there is an early predominant role of IGF1R inhibition on tumor growth reduction. IGF1R binder treated tumors did not show significant reductions in tumor weight. The combo treated tumors were smaller than IGF1R binder only and the vehicles, however, not smaller than VEGFR2 binder alone. It should be noted that the larger tumor weights on the bevacizumab treated groups is related to the lack of effect of bevacizumab and, also, as a result to the initial larger tumor volume of this group at the randomization time.

Note that the tumors used for casting were dissected out and weighed after perfusion and injection of the casting medium. However, due to the minor differences in specific weights after resin perfussion the error is considered to be negligible. Tumors used for corrosion casting were collected two weeks after transplantation and treatment.

(III) 2D Evaluation of Microvessel Density

The vehicle treated tumors show a 5.54% vascular surface area, a significantly higher vessel fraction than all 10Fn3 treated groups:
Vehicle-treated tumors contain nearly five times more vessels per area than the VEGFR2 binder treated tumors.
When subtracting the necrotic areas and calculating only the mean vessel surface area in viable tissue, the differences between the different treatments and vehicle are statistically significant.
Due to the fact that the necrotic areas contain by far less vessels, the percentual vessel surface area increases proportionally.
IGF1R binder had the lowest effect whereas VEGFR2 and Combo resulted in best effects.
VEGFR2/IGF1R binder showed an intermediate phenotype between VEGFR2 binder and IGF 1R binder with no statistically significant differences compared to IGF1R binder.
No significant differences were reached between VEGFR2 binder and the combination. From that it can be concluded that inhibition of VEGFR2 had a dominant role on microvascular reduction in the presence of IGFR inhibition with no additive combinatorial effect in this early stage.
These results suggest that the drugs do not only reduce the total amount of newly formed vessels—the treated tumors were in general smaller than the controls—but also that within identical tumor volumes or sectional surfaces there are significantly fewer vessels than in the controls.

(IV) Tumor Cast Vascular Volume

A large variation was observed in casts derived from mice treated with IGF1R binder. These results suggest that though a reduction on microvascular density was observed by CD31 staining, the residual vasculature is still largely permeable accounting for the inability to “cast” these vessels:
The combo, VEGFR2 binder, and VEGFR2/IGF1R binder groups showed significantly smaller vascular volumes than vehicle. These results suggest a dominant role of VEGFR2 inhibition on the reduction of microvascular density with the highest impact observed from the combo group.
These results are in line with the 2D microvascular density data.
The highest absolute vessel volumes were seen in the vehicles and the bevacizumab group.

(V) Microvascular Architecture: Qualitative Aspects

Treatments, CompA=IGF1R binder, CompB=VEGFR2/IGF1R binder.
Vehicle: The vehicle treated tumors display a chaotic arrangement with high vascular densities and no hierarchy of the vascularity.
The diameters of the individual vessel segments for the binder treatments show only little variations, whereas the controls are characterized by large caliber, sinusoidal vessel networks with frequent vessel diameter changes.
VEGFR2 binder: The vasculature of VEGFR2 binder treated tumors is characterized by:
   A lower vessel density with normal diameters
   A microvessel architecture resembling more the normal, autochthonous vascular architecture
Bevacizumab: Both the VEGFR2 binder and bevacizumab treated tumors showed a remodeling of the vascular patterns. A higher vessel density was observed in bevacizumab treated tumors, in accordance with the larger size of these tumors. The architecture of these tumors is closer to the observed on VEGFR2 binder treated tumors, though in tumor size they were closer to the vehicle.
IGF1R binder: Appearance of vessels is very close to the controls with all typical features of tumor vascular architecture. The total amount of newly formed vessels seems lower, but these tumors can be hardly discerned from the controls based on their architecture.
VEGFR2/IGF1R binder: Tumors treated with VEGFR2/IGF1R binder are mostly similar to the ones treated with VEGFR2 binder. VEGFR2/IGF1R binder treatment seems to impact the vasculature on a heterogeneous fashion, however, the majority of the tumors show qualitatively properties of the VEGFR2 binder tumors. Phenotypically, there is no striking “differentiation” from VEGFR2 binder.
Combo: Phenotype of tumor vasculature is very similar to that of VEGFR2/IGF1R binder except that vessels diameter where it was most similar to VEGFR2 binder (VEGFR2/IGF1R binder had an intermediate range between those observed for VEGFR2 binder and vehicle.

(VI) Microvascular Architecture: Quantitative Data

The vessel diameters are the lowest for the combo, VEGFR2 binder, and bevacizumab: less than half of vessel diameters for vehicle-treated tumors.
The increase on intervascular distances is more apparent on combo, VEGFR2 binder, groups vs. vehicle, and less apparent on IGF1R binder and VEGFR2/IGF1R binder groups vs. vehicle. These results parallel the qualitative finding of vascular remodeling in these groups.
Tumors treated with VEGFR2/IGF1R binder had an intermediate phenotype for vessel diameters between the range observed for VEGFR2 binder and vehicle. These results suggest that IGF1R inhibition may influence events required for tumor vascular remodeling differently from VEGFR2.
Bevacizumab-treated tumors presented the smallest intervascular distance parameter in accordance with their higher vascular density compared to any of the binder groups or the vehicle control.

Although the invention has been described in some detail by way of illustration and example for purposes of clarity and understanding, it will be apparent that certain changes and modifications may be practiced within the scope of the appended claims.

What is claimed is:

1. A method for increasing endothelial cell apoptosis under hypoxic conditions in a patient undergoing a treatment that comprises an IGF1R inhibitor, said method comprising administering to said patient a VEGFR2 inhibitor.

2. The method of claim 1 wherein said IGF1R inhibitor and said VEGFR2 inhibitor are co-administered to said patient.

3. The method of claim 1 wherein at least one of said IGF1R inhibitor and said VEGFR2 inhibitor is not derived from 10Fn3.

4. A method for identifying a molecule for the treatment of a proliferative disease comprising the steps of:
(i) identifying an IGF1R inhibitor;
(ii) identifying a VEGFR2 inhibitor; and
(iii) combining the properties of said first agent and send second agent in a molecule that is effective for the treatment of said proliferative disease.
